(19) United States
(12) Patent Application Publication
(10) Pub. No.: US 2006/0240116 A1
(43) Pub. Date: Oct. 26, 2006

(54) Bioactive factors in wound healing topical compositions and methods

(76) Inventor: David Jolley, Tucson, AZ (US)

Correspondence Address: TRASK BRITT, P.O. BOX 2550, SALT LAKE CITY, UT, 84110, US

(21) Appl. No.: 11/112,968

(22) Filed: Apr. 22, 2005

(51) Int. Cl. A61K 35/20; A61K 31/74; A61K 38/43

(52) U.S. Cl. 424/535; 424/78.02; 424/94.1

(57) ABSTRACT

A topical composition for treating a cutaneous wound includes whole colostrum or one or more growth factors, as well as a base by which the whole colostrum or one or more growth factors are carried. The topical composition may also include, without limitation, immunomodulators, lactoferrin, enzymes, vitamins, minerals, amino acids, or combinations thereof. Other components may also be included in the topical composition, including, but not limited to, antiseptics, antimicrobial agents, and anesthetics. When applied to a wound, the bioactive topical composition promotes healing of the treated wound.

### BACKGROUND OF THE INVENTION

1. Field of the Invention

The present invention relates generally to topical compositions and, more specifically, to topical compositions that promote the healing of cutaneous lesions. In particular, the present invention relates to topical bioactive compositions that include one or more growth factors (GF) (e.g. epidermal, fibroblast, insulin-like I&II, transforming α&β, platelet-derived, nerve), immune factors (e.g. immunoglobulins, lactoferrin, cytokines, proline-rich polypeptide, lactalbumins) enzymes (e.g. lysozyme, peroxidase, proteases, xanthine oxidase) micro-and macro-nutrients (e.g. carbohydrates, amino acids, vitamins, minerals) or combinations thereof, as are present in colostrum.

2. Background of Related Art

Dairy products, including milk, and whey are known to include a variety of components, termed “growth factors,” that facilitate cell growth and repair, and therefore, may be useful in enhancing the rates at which wounds heal.

Methods for purifying growth factors from milk and whey products are disclosed in U.S. Pat. Nos. 5,866,418, 6,194,208, 6,319,522, and 6,447,808, as well as in U.S. Patent Application Publication US 2003/0059477, all of which have been assigned to GroPep, Ltd., of Thebarton South, Australia, and thus, are collectively referred to hereinafter as “the GroPep Patents.” Specifically, the GroPep Patents disclose processes for purifying various growth factors from purportedly undesirable acidic components of milk or whey, including, among others, immunoglobulins (antibodies), albumin, and lactoferrin. As U.S. Pat. No. 5,866,418 indicates, these purified growth factors were initially used as a substitute for fetal bovine serum (a fetal calf blood product) in facilitating the growth of cells artificially, in cell cultures, or in vitro. Apparently, the named inventors of the processes and compositions disclosed in the GroPep Patents believed that the acidic components of milk and whey interfered with cell growth.

The GroPep Patents also suggest that the disclosed purified growth factors may be useful for other purposes, including treatment of wounds, diseases, and gastrointestinal disorders, including ulcers. To this end, the GroPep Patents disclose that growth factors that have purified from milk or whey may be combined with pharmaceutically acceptable carriers, such as compositions that are configured for enteral, or oral, administration or parenteral administration (e.g., topically, by injection, etc.).

As noted, various acidic components of milk and whey, including immunoglobulins, albumin, and lactoferrin, are eliminated by the purification processes that are disclosed in the GroPep Patents. Antibodies are known to provide a defense against pathogens. Lactoferrin, a substance present in tears, has known antimicrobial properties. Albumin is a source of a variety of essential amino acids, which are the building blocks of proteins, which, in turn, are important to the growth and repair of cells. Of course, a variety of other constituents of milk and whey that are important to the generation and growth of new cells and, thus, the process of healing a wound or other injury are also removed as the processes disclosed in the GroPep Patents are used to remove acidic components from neutral and basic growth factors. Thus, the processes that are disclosed in the GroPep Patents potentially diminish the effectiveness of the resulting products in treating wounds, diseases, and gastrointestinal injuries.

Another substance that is secreted by the glands of a lactating mammal, colostrum, is also known to include relatively high concentrations of a variety of growth factors. Colostrum is the first lacteal secretion of a female mammal (e.g., primate, bovine, porcine, etc.) following the birth(s) of her offspring. This chemically-complex fluid is produced weeks before parturition, stored in the mammary glands, and delivered to the suckling newborn for up to 72 hours after birth. As colostrum is a newborn's first food, it has higher concentrations of many of the bioactive factors that sustain life and promote growth. Passive immunity is also transferred to the neonate by this colostrum.

Bovine (cow) colostrum has been used in a wide variety of orally consumable dietary supplements, and research indicates usefulness in treating gastrointestinal conditions. The marketing of these supplements purport to supporting proper digestive function and promoting a healthy immune system

The inventor is unaware of any use of neither colostrum or compositions that include acidic components of colostrum utilized in the treatment of external wounds, nor any topical compositions that include whole colostrum or acidic components thereof.

### SUMMARY OF THE INVENTION

The present invention includes topical compositions and methods for using topical compositions.

A topical composition according to the present invention includes a base, or carrier, and one or more bioactive wound reparative components. The base is formulated to deliver the healing promotion component (bioactive factors) to a wounded area (e.g., an open lesion) on the skin or other tissues of a subject, while the restorative component promotes repair of the wounded area.

Exemplary bases that may be used in the inventive topical composition include gels (e.g., hydrogels), salves, lotions, creams, and the like, as are known to be useful in topical compositions. In addition to carrying the one or more bioactive factors of the topical composition, the base may be formulated for other therapeutic purposes, including moisturization, prevention of infection, and promotion of healing.

The healing promotion component of a topical composition of the present invention may, by way of nonlimiting example, include whole colostrum, which is the chemically-complex first lacteal secretions of a female mammal (e.g., primate, bovine, porcine, etc.) following the birth(s) of her offspring. This colostrum is expelled by the mammary glands, and delivered to the suckling newborn for up to 72 hours after birth. As used herein, the term “whole colostrum” refers to whole liquid colostrum, as obtained from a lactating mammal, as well as liquid and solid (e.g., powder) concentrates of colostrum.

Alternatively, the bioactive factors for wound healing of a topical composition according to the present invention may include one or more of the various components of whole colostrum, including, without limitation, growth factors, lactoferrin, immunomodulators, enzymes, micro- and macro-nutrients.

In addition to the base and healing promotion component (i.e., bioactive factors), a topical composition that incorporates teachings of the present invention may include other components. Examples of such components include, but are not limited to aloe vera or a derivative thereof (e.g., acemannan), colloidal or ionic silver, povidone-iodine, or any other component that may provide the topical composition with a desired characteristic.

Methods for treating cutaneous lesions are also within the scope of the present invention. In such methods, a topical composition according to the present invention is applied to a wound at least once. Such a topical composition may also be applied multiple times, even periodically. Additionally, a dressing of a known type may be applied over the wound and the topical composition with which the wound has been treated.

Other features and advantages of the present invention will become apparent to those of ordinary skill in the art through consideration of the ensuing description and the appended claims.

### DETAILED DESCRIPTION

A topical composition of the present invention includes a base, or carrier, and a healing promotion component.

The base, which is formulated to deliver the healing promotion component to a wounded area (e.g., an open lesion) on skin or other tissues of a subject, may include any suitable topical substance known in the art. It is currently preferred that a composition that is suitable for use as a wound dressing or that is otherwise suitable for application to wounds be employed as the base of a topical composition according to the present invention. Exemplary bases include, without limitation, gels, ointments, salves, lotions, and creams.

In an exemplary embodiment of topical composition that incorporates teachings of the present invention, the base includes a gel in which the liquid constituent is water (i.e., hydrogel) and may include a variety of forms of different polymers which lend structural stability to the product. A definition of a hydrogel has been stated as ‘a quasi-solid produced from a hydrocolloid (hydrosol), when the disperse solid phase is rendered continuous by some stimulus. Hydrogels of various types have been utilized extensively for the treatment of chronic and acute wounds for greater than 20 years. Various hydrogels are commercially available which are suitable for use as the base, including, but not limited to, CARRASYN® hydrogel wound dressing, manufactured by Carrington Laboratories, Inc., of Irving, Tex., CURASOL® gel wound dressing, manufactured by Healthpoint, Ltd., of San Antonio, Tex., and TRIAD hydrophilic wound dressing, manufactured by Coloplast Corp. of Marietta, Ga.

In another exemplary embodiment of topical composition according to the present invention, the base includes an enzymatic debridement ointment (i.e., collagenase) such as the papain-urea ointment marketed as ACCUZYME® by Healthpoint, Ltd., a DFB Pharmaceuticals Company, of Fort Worth, Tex., or COLLAGENASE SANTYL®, available from Ross Products Division of Abbott Laboratories, Inc., Abbott Park, Ill.

As a topical composition that incorporates teachings of the present invention is to be used to treat wounds, the base, healing promotion component, or both may be sterile (by way of known, acceptable sterilization techniques) or aseptic.

Bioactive factors of a topical composition according to the present invention, which promotes healing of the wounded area, are carried by the base. For example, the healing promotion component may be dispersed throughout the base. The healing promotion component may be dispersed throughout the base in any suitable fashion, such as by dissolution in at least a part of the base (e.g., hydrophilic, or “water-loving,” constituents of the healing promotion component being dissolved in hydrophilic portions of the base; or hydrophobic, or “water-hating,” constituents of the healing promotion component being dissolved in hydrophobic portions of the base), particle dispersion, emulsion (i.e., by way of surface-acting agents, or “surfactants”), or in any other suitable manner. This may include, but is not limited to, applying the bioactive factors directly to the wound, followed by a covering of the appropriate base.

TABLE 1 identifies, without limitation, a variety of bioactive factors, including but not limited to growth factors and cytokines, that may be employed individually or in any combination in a topical composition that incorporates teachings of the present invention, and lists the processes by which they may facilitate wound repair.

TABLE 1

| Process | Growth Factors/Cytokines Involved |
|---|---|
| Neutrophil | TGF-β, MCP-1, MIP2/GRO-α, IL-8, IL-6, |
| infiltration | IL-10(−) |
| Macrophage | TGF-β, MCP-1, MIP1-α, IL-10(−) |
| infiltration | |
| Angiogenesis | VEGF-A, PLGF, FGF2, Angiopoietins, HGF, Cyr61, |
| | MCP-1, IL-8, GRO-α, GM-CSF, IP-1O(−) |
| Fibroplasia | PDGF, TGF-β, CTGF, GM-CSF, IGFs |
| Matrix | FGF2, IGF-1, NGF, TGF-β, Activin, MCP-1, |
| deposition | CTGF, Cyr61 |
| Scarring | IGF-1, TGF-β, Activin, CTGF, IL-6, IL-10(−) |
| Reepitheliali- | FGF2, FGF7, FGF10, EGF, TGF-α, HB-EGF, NDF, |
| zation | IGFs, NGF, Activin, MCP-1, IL-6, GM-CSF, Leptin, |
| | TGF-β(−), BMP-6(−), IP-10(−) |

Reproduced from Werner S, Grose R, in Regulation of Wound Healing by Growth Factors and Cytokines. Physiol Rev. 2003

An exemplary wound reparative component that may be included in a topical composition of the present invention includes whole bovine colostrum. The colostrum formulation may be heat-pasteurized for additional purity. Whole bovine colostrum is available in powder form from a variety of sources, including NEW ZEALAND GOLD colostrum, available from Metafoods of Cottonwood, Ariz., PRIME LIFE colostrum, available from Jarrow Formulas of Los Angeles, Calif. Of course, colostrum from other mammalian sources (e.g., ovine, porcine) may be used in addition to or in place of bovine colostrum. The colostrum may be included in the base in any suitable, therapeutically effective concentration. By way of example only, the colostrum may be present in the base in a concentration of about 10 milligrams (mg) to about 1,000 mg of the whole bovine colostrum product per cubic centimeter (cc), or milliliter (mL), of the base.

TABLE 2 provides an example of bioactive factors that may be present in colostrum:

TABLE 2

| Constituents | Examples |
|---|---|
| Growth Factors | EGF, FGF, IGF-1 IGF-2, TGF-α, TGF-β, PDGF, |
| | VEGF, NGF, CTGF, Growth Hormone, Insulin |
| Immunomodulators | IgG, IgM, IgE IgA, SigA, Lactoferrin, |
| | Transferrin, Protease, PRP, IL-6, IL-8, |
| | IL-10, IF-γ, Lymphkines, Lysozyme, |
| | C3, C4, TNF |
| Vitamins and | Vitamins B1, B2, B6, B12, E, A, C, Folic |
| Other Nutrients | Acid, Panthothenic Acid, Beta-carotene, |
| | Glycogen, Retinoic Acid |
| Minerals | Calcium, Chromium, Iron, Magnesium, |
| | Phosphorous, Potassium, Sodium, Zinc |
| Essential | Isoleucine, Leucine, Histidine, Methionine, |
| Amino Acids | Lysine, Threonine, Phenylalanine, Valine, |
| | Tryptophan |
| Nonessential | Argining, Cystine, Glutanic Acid, Alanine, |
| Amino Acids | Tyrosine, Glycine, Proline, Aspartic Acid, |
| | Serine |
| Additional | β-2 microglobulin, Haemopexin, Haptoglobulin, |
| Factors | Lactoperoxidase, Orotic Acid, Peroxidase, |
| | Xanthine Oxidase, Glycoproteins |

Key: (−) = Negative regulation, TGF = Transforming Growth Factor, MCP = Macrophage Chemoattractant Protein, MIP = Macrophage Inflammatory Protein, GRO = Growth-Related Oncogene, IL = Interleukin, VEGF = Vascular Endothelial Growth Factor, PLGF = Placenta Growth Factor, FGF = Fibroblast Growth Factor, HGF = Hepatocyte Growth Factor, Cyr61 = Cysteine-Rich 61, GM-CSF = Granulocyte-Macrophage Colony Stimulating Factor, IP = Interferon-γ-Inducible Protein, PDGF = Platelet-Derived Growth Factor, CTGF = Connective Tissue Growth Factor, IGF = Insulin-like Growth Factor, NGF = Nerve Growth Factor, EGF = Epidermal growth Factor, HB-EGF = Heparin-Binding Epidermal Growth Factor, NDF = Neu Differentiation Factors, BMP = Bone Morphogenetic Proteins, Ig = Immunoglobulin, PRP = Proline-Rich Polypeptide, C = Complement, IF = Interferon-γ

As an alternative to colostrum, the healing promotion component of a topical composition that incorporates teachings of the present invention may include one or more bioactive factors that are present in colostrum.

For example, one or more growth factors may make up at least a part of the healing promotion component. Growth factors are proteins that facilitate the growth of new cells, and repair of damaged cells. They may be purified from natural sources or manufactured using well-known recombinant gene-expression technologies. Examples of natural, purified growth factors (GF) that may be included in the healing promotion component includes epidermal GF, fibroblast GF, transforming GF-α and β, insulin-like GF I and II, platelet-derived GF, nerve GF, connective tissue GF, or the like. Currently, the only FDA approved recombinant growth factor therapy for the healing of cutaneous wounds is REGRANEX® (becaplermine), commercially available from Johnson & Johnson of New Brunswick, N.J. REGRANEX® contains 100 micrograms of recombinant human platelet-derived growth factor per gram of hydrogel. Other recombinant growth factors are currently in the various stages of research and development.

Alternatively, or in addition to one or more growth factors, the healing promotion component of a topical composition of the present invention may include lactoferrin (which is present in human tears and has known antimicrobial properties), immunoglobulins, hormones, insulin, enzymes, amino acids, vitamins, minerals, other components of colostrum, or combinations thereof.

Other components of colostrum that may be included in a topical composition of the present invention include, but are not limited to, immunoglobulins, albumin, and other acidic components. These components may be present in such a topical composition in a concentration that equals or exceeds their normal concentrations in whey (i.e., at least about 0.5%).

In addition to the base and healing promotion component, a topical composition that incorporates teachings of the present invention may include one or more antiseptic or antimicrobial components, which may prevent infection of the treated wound. Any known antiseptic or antimicrobial agent which is suitable for treating superficial wounds may be included in a topical composition of the present invention as an antiseptic or antimicrobial component thereof. Antimicrobial agents (e.g., antibacterial, antiviral, antifungal, antiparasitic, etc.) are known to exist within colostrum. Immunoglobulins (antibodies) are another example of an antimicrobial component that may be found with colostrum. Ionic silver and povidone-iodine, which are known to be lethal to bacteria, viruses, and fungi, are other examples of antimicrobial components that may be used in conjunction with colostrum. Of course, the concentration of antiseptic or antimicrobial components in such a topical composition may be effective for preventing infection or treating infection, as known in the art.

A topical composition of the present invention may, optionally, include one or more anesthetic components, as known in the art. The concentration of anesthetic or anesthetics in such a topical composition may, of course, be sufficient to reduce or eliminate pain caused by a treated wound, as known in the art.

Another aspect of the present invention includes methods for treating superficial (e.g., skin) wounds. Wound treatment may include application of a topical composition that incorporates teachings of the present invention to a wound. By way of nonlimiting example: neurotrophic (i.e., diabetic foot) ulcers, pressure (decubitus) ulcers, venous insufficiency or stasis ulcers, dehisced surgical wounds, cuts, abrasions, skin conditions associated with peristomal care, radiation dermatitis, and first and second degree burns, including sunburn and windburn, may be treated with a topical composition of the present invention.

TABLE 3 identifies exemplary core healing principles and the bioactive factors that may be useful in addressing and improving each of these core healing principles:

TABLE 3

| | Core Healing | |
|---|---|---|
| | Principles* | Colostrum Wound Gel Solutions |
| MACROscopic | Infection | Lactoferrin, Cytokines, |
| Wound | | Lysozyme |
| Enviroment | Metabolic Control | Insulin, Vitamins, Minerals, |
| | and Nutrition | Amino Acids |
| | Immune Status | Immunoglobulins and |
| | | Immunomodulators |
| | Perfusion | Vascular Endothelial Growth |
| | | Factor |
| | Tissue Moisture | Hydrogel Suspension for Moist |
| | Balance | Wound Healing |
| | Necrosis | Autolytic Enzymes |
| MICROscopic | Bioburden | Cytokines, Lactoferrin, Lysozyme |
| Wound | Growth factor | Supplies TGFα&β, IGF-I&II, |
| Enviroment | Deficiencies | VEGF, NGF, PDGF, FGF |
| | Abnormal | VEGF |
| | Microcirculation | |
| | Proliferative | Macro- and Micro-nutrients |
| | Capacity | Supplied to Cells for Growth |
| | Excessive | Cytokines and Interleukins |
| | Inflammatory | |
| | Mediators | |

*Adapted from: Johnson & Johnson Wound Management Worldwide, A Division of Ethicon, 2004.

In such methods, a topical composition according to the present invention is applied to the wound at least once. Such a topical composition may also be applied multiple times, even periodically. Additionally, a dressing of a known type may be applied over the wound and the topical composition with which the wound has been treated.

In the following EXAMPLES, details are provided on topical compositions that incorporate teachings of the present invention, as well as on treatment methods according to the present invention:

### EXAMPLE 1

A composition including about 100 mg NEW ZEALAND GOLD colostrum powder, which includes about 10 mg lactoferrin, in each cubic centimeter of hydrogel was prepared.

### EXAMPLE 2

The composition from EXAMPLE 1 was applied to patients' wounds in outpatient wound clinics. A variety of different types of wounds were treated including: diabetic, pressure, and venous stasis ulcerations, dehisced surgical incisions, cutaneous eruptions secondary to SLE and localized amyloidosis, lesions secondary to a Hobo spider bite (tegenarism), superficial cuts, abrasions and lacerations. An initial study of nine females and 17 males (n=26) were treated for a total of 36 lower extremity lesions (18 patients with a single lesion, six with 2 lesions, and 2 patients with 3 lesions) were treated over a period of 31 weeks by the physicians and staff at two treatment centers. If a patient had greater than one wound, one lesion was treated with the topical composition of EXAMPLE 1, and the other wound(s) treated with hydrogel lacking colostrum and lactoferrin. The rate at which the wounds treated with the bioactive factors found in EXAMPLE 1 progressed, was compared with the rate at which the similar, control wound(s) at a generally corresponding location progressed. In all cases, accelerated wound healing was noted in the lesions treated with the bioactive factors found in EXAMPLE 1

Use of the bioactive gel was in concert with standard-of-care treatment specific to the etiology of the lesion (e.g., compressive dressings for venous ulcerations, offloading for diabetic foot ulcerations, etc). All patients were seen 3-7 days between visits, until complete wound healing was noted.

The total length of the study continued for 31 weeks, with patients being treated to full wound recovery. In all subjects with multiple lesions, the wound that was treated with the bioactive colostrum-hydrogel composition healed at a faster rate than the wound(s) treated with hydrogel lacking colostrum and lactoferrin. Additionally, in some cases photographs were used to track progress of the healing process.

Use of the bioactive gel was in concert with standard-of-care treatment specific to the etiology of the lesion (i.e. compressive dressings for venous ulcerations, offloading for diabetic foot ulcerations, etc). Average time to complete healing for all patients: 58.6 days (shortest: one week, longest: 136 days), as indicated by TABLE 4:

TABLE 4

| | Patient | | |
|---|---|---|---|
| Condition | # | | |
| Diabetic Foot (Neurotrophic) | 1 | 17 | days |
| Ulcerations | 2 | 18 | days |
| | 3 | 31 | days (two ulcers) |
| | 4 | 34 | days |
| | 5 | 47 | days |
| | 6 | 57 | days |
| | 7 | 62 | days |
| | 8 | 70 | days |
| | 9 | 84 | days (two ulcers) |
| | 10 | 93 | days (two ulcers) |
| Venous Stasis Ulcerations | 11 | 14 | days |
| | 12 | 14 | days |
| | 13 | 23 | days |
| | 14 | 28 | days |
| | 15 | 28 | days |
| | 16 | 31 | days (two ulcers) |
| | 17 | 49 | days |
| | 18 | 51 | days (two ulcers) |
| | 19 | 67 | days (two ulcers) |
| Pressure (Decubitus) | 20 | 42 | days |
| Ulcerations | 21 | 136 | days (three ulcers) |
| Dehisced Surgical Wounds | 22 | 9 | days |
| | 23 | 22 | days |
| | 24 | 34 | days |
| Cutaneous Eruption | 25 | 63 | days |
| (secondary to SLE) | | | |
| Cutaneous Eruption | 26 | 32 | days (three ulcers) |
| (secondary to Localized | | | |
| Amyloidosis) | | | |

The longest course of treatment (total of 136 days) was given to a male patient with three decubiti ulcerations of the left foot. These wounds had been present for 6 months prior to enrollment in the study. All three wounds were closed at day 136, and have remained so at each follow-up visit.

The faster rate of healing was noted in a venous stasis ulceration of the lower leg. This superficial lesion had been present for approximately 3 weeks prior to enrollment into the study. The bioactive formulation of EXAMPLE 1 was applied to the wound followed by a multi-layer compression dressing. At the one-week follow-up visit, complete healing of the stasis ulceration was noted.

Only one of the treated patients exhibited any noted complications. The complications were due to inadequate cleansing of the wound prior to application of the topical composition of EXAMPLE 1. This inadequate cleansing caused a superficial infection, which was treated with oral antibiotics. Treatment of the wound with the topical composition of EXAMPLE 1 continued while the patient was receiving the oral antibiotics. The infection subsided, and the patient continued on to complete healing of the ulceration.

### EXAMPLE 3

A topical composition including about 100 mg NEW ZEALAND GOLD colostrum powder, which includes about 10 mg lactoferrin, was incorporated into 1 cc of a collagenase enzymatic debridement ointment.

### EXAMPLE 4

The topical composition of EXAMPLE 3 was applied to a dried eschar (i.e., a scab or slough on the skin) that occurred on an ischemic, non-healing surgical wound, as well as on pressure (i.e., decubitus) ulcers.

The topical composition of EXAMPLE 3 was applied to one patient, with accelerated rate of debridement of necrotic tissue, as compared to previous applications of enzymatic debridement ointments without colostrum added. This patient was lost to follow-up as he moved out of state.

Although the foregoing description contains many specifics, these should not be construed as limiting the scope of the present invention, but merely as providing illustrations of some of the presently preferred embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

1. A topical composition for application to a wound, comprising:
a pharmaceutically acceptable carrier; and
whole colostrum.

2. The topical composition of claim 1, wherein the pharmaceutically acceptable carrier comprises a hydrogel.

3. The topical composition of claim 1, wherein the pharmaceutically acceptable carrier comprises a collagenase.

4. The topical composition of claim 1, further comprising:
at least one of an anesthetic, an antiseptic, and an antimicrobial.

5. The topical composition of claim 1, further comprising:
lactoferrin.

6. A topical composition for application to a wound, comprising:
a pharmaceutically acceptable carrier;
at least one growth factor; and
at least one acidic component present in a therapeutically effective concentration.

7. The topical composition of claim 6, wherein the pharmaceutically acceptable carrier comprises a hydrogel.

8. The topical composition of claim 6, wherein the pharmaceutically acceptable carrier comprises a collagenase.

9. The topical composition of claim 6, further comprising:
at least one of an anesthetic, an antiseptic, and an antimicrobial.

10. The topical composition of claim 6, further comprising:
lactoferrin.

11. The topical composition of claim 6, wherein the at least one growth factor comprises at least one of epidermal growth factor, fibroblast growth factor, insulin-like growth factor I, insulin-like growth factor II, growth factor-α, transforming growth factor-β, platelet-derived growth factor, nerve growth factor, gonadotropin-releasing hormone, growth hormone, insulin, prolactin, and REGRANEX.

12. The topical composition of claim 6, wherein the at least one acidic component comprises at least one of immunoglobulins and albumin.

13. The topical composition of claim 6, wherein the at least one acidic component is present in at least the same concentration the at least one acidic component is present in whey.

14. A topical composition for application to a wound, comprising:
a hydrogel; and
at least one growth factor.

15. The topical composition of claim 14, wherein the at least one growth factor is dispersed throughout the hydrogel.

16. The topical composition of claim 14, wherein the at least one growth factor comprises at least one of epidermal growth factor, fibroblast growth factor, insulin-like growth factor I, insulin-like growth factor II, growth factor-α, transforming growth factor-β, platelet-derived growth factor, nerve growth factor, gonadotropin-releasing hormone, growth hormone, insulin, prolactin, and REGRANEX.

17. A topical composition for application to a wound, comprising:
a collagenase; and
at least one growth factor.

18. The topical composition of claim 17, wherein the at least one growth factor is dispersed throughout the hydrogel.

19. The topical composition of claim 17, wherein the at least one growth factor comprises at least one of epidermal growth factor, fibroblast growth factor, insulin-like growth factor I, insulin-like growth factor II, growth factor-α, transforming growth factor-β, platelet-derived growth factor, nerve growth factor, gonadotropin-releasing hormone, growth hormone, insulin, prolactin, and REGRANEX.

20. A method for treating a wound, comprising:
applying a composition including whole colostrum to the wound.

21. The method of claim 20, wherein applying comprises applying a composition further including a moisturizing component to the wound.

22. The method of claim 21, wherein applying comprises applying a composition with the whole colostrum dispersed throughout the moisturizing component.

23. The method of claim 20, wherein applying comprises applying a composition further including lactoferrin to the wound.

24. The method of claim 20, wherein applying comprises applying a composition further including a collagenase to the wound.

25. A method for treating a wound, comprising:
applying a composition including at least one growth factor and at least one of a hydrogel, a collagenase, and an acidic component to the wound.
